(19) United States
(12) Patent Application Publication
(10) Pub. No.: US 2011/0198989 A1
(43) Pub. Date: Aug. 18, 2011

(54) NOVEL ORGANIC COMPOUND AND ORGANIC LIGHT-EMITTING ELEMENT USING THE SAME

(75) Inventors: Yosuke Nishide, Kawasaki-shi (JP); Takayuki Horiuchi, Tokyo (JP); Jun Kamatani, Tokyo (JP); Naoki Yamada, Inagi-shi (JP); Akihito Saitoh, Yokohama-shi (JP)

(73) Assignee: CANON KABUSHIKI KAISHA, Tokyo (JP)

(21) Appl. No.: 13/026,039

(22) Filed: Feb. 11, 2011

(30) Foreign Application Priority Data
Feb. 15, 2010 (JP) 2010-030453

(51) Int. Cl. H01J 1/62; C07C 13/72

(52) U.S. Cl. 313/504; 585/27

(57) ABSTRACT

Provided are an organic compound expressed by General Formula (1) and an organic light-emitting element using the organic compound:

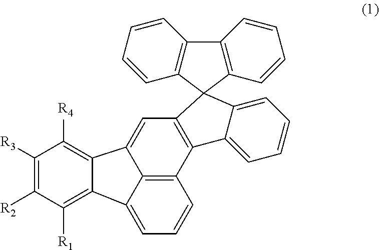

wherein R₁ to R₄ are each hydrogen, an alkyl group or an aryl group.

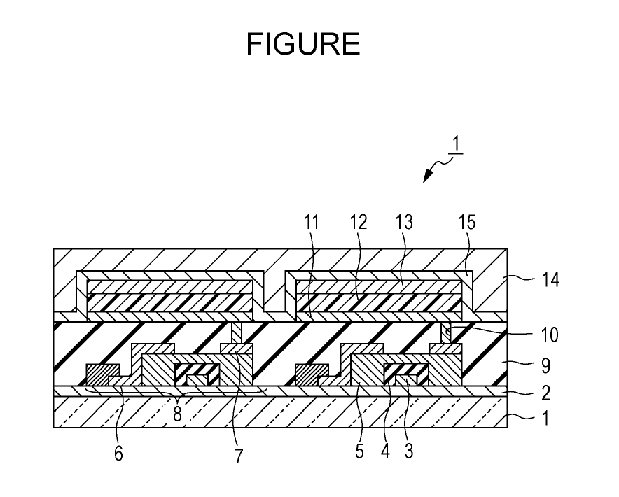

### BACKGROUND OF THE INVENTION

1. Field of the Invention

The present invention relates to a novel organic compound and an organic light-emitting element using the same.

2. Description of the Related Art

An organic light-emitting element includes a pair of electrodes and an organic compound layer disposed between the pair of electrodes. The electrodes inject carriers to the organic compound layer to excite the organic compound in the organic compound layer. When the organic compound returns to the ground state, light is emitted.

Organic light-emitting elements are called organic electroluminescence elements or organic EL elements. Various organic compounds have been actively developed. Creation of a novel organic compound is important for providing a high-performance organic light-emitting element. It is known that the quantum yield of an organic compound in a luminescent layer highly depends on the luminous efficiency of the organic light-emitting element, and that a high quantum yield leads to a high luminous efficiency.

In Japanese Patent Laid-Open No. 10-189248, it is disclosed that an organic light-emitting element using a fluoranthene derivative emits blue light.

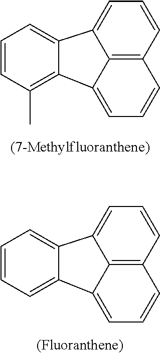

However, fluoranthene has a low quantum yield of 0.35, and organic light-emitting elements using fluoranthene exhibit low luminous efficiencies accordingly.

### SUMMARY OF THE INVENTION

The present invention provides a novel organic compound that includes a basic skeleton not having a rotatable bond, and consequently has a high quantum yield.

According to an embodiment of the present invention, an organic compound is provided which is expressed by the following General Formula (1):

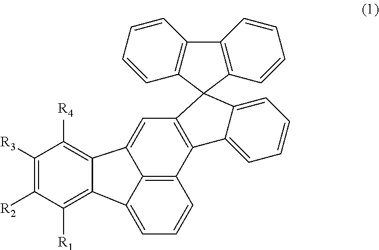

In the General Formula (1), R₁ to R₄ are each selected from the group consisting of hydrogen, alkyl groups and aryl groups. The alkyl groups consist of methyl, ethyl, n-propyl, isopropyl, n-butyl, isobutyl, sec-butyl, and tert-butyl. The aryl groups consist of phenyl, biphenyl, terphenyl, naphthyl, phenanthrenyl, anthracenyl, fluoranthenyl, benzofluoranthenyl, and fluorenyl. The alkyl groups and the aryl groups may have a substituent selected from the group consisting of alkyl, aralkyl, aryl, heterocycle, amino, alkoxyl, cyano, and halogens.

The embodiment of the present invention can provide a novel organic compound that includes a basic skeleton not having a rotatable bond, and consequently has a high quantum yield. In addition, an organic light-emitting element using the organic compound can exhibit a high luminous efficiency and a high durability.

Further features of the present invention will become apparent from the following description of exemplary embodiments with reference to the attached drawing.

### BRIEF DESCRIPTION OF THE DRAWINGS

The FIGURE is a schematic sectional view of a display device including organic light-emitting elements and switching elements connected to the respective organic light-emitting elements.

### DESCRIPTION OF THE EMBODIMENTS

The organic compound according to an embodiment of the present invention is expressed by the following General Formula (1):

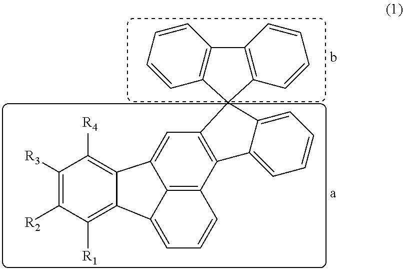

In General Formula (1), R₁ to R₄ are each selected from the group consisting of hydrogen, alkyl groups and aryl groups. The alkyl groups consist of methyl, ethyl, n-propyl, isopropyl, n-butyl, isobutyl, sec-butyl, and tert-butyl. These alkyl groups act as sterically hindering groups to inhibit molecular aggregation. Among these groups, tert-butyl is effective.

The aryl groups consist of phenyl, biphenyl, terphenyl, naphthyl, phenanthrenyl, anthracenyl, fluoranthenyl, benzofluoranthenyl, and fluorenyl. These aryl groups act as sterically hindering groups to inhibit molecular aggregation. This effect of the aryl groups is stronger than that of the alkyl groups. The aryl groups increase the conjugation length, and accordingly increase the emission wavelength. In particular, the phenyl, biphenyl, naphthyl, fluorenyl, and benzofluoranthenyl groups can increase the conjugation length to change the emission wavelength and enhance the quantum yield of the compound.

The alkyl groups and the aryl groups may have a substituent. Examples of the substituent include: alkyl groups, such as methyl, ethyl, and propyl; aralkyl groups, such as benzyl and phenethyl; aryl groups, such as phenyl, biphenyl, naphthyl, phenanthrenyl, fluorenyl, pyrenyl, and fluoranthenyl; heterocycle groups, such as thienyl, pyrrolyl, and pyridyl; amino groups, such as dimethylamino, diethylamino, dibenzylamino, diphenylamino, ditolylamino, and dianisolylamino; alkoxyl groups, such as methoxyl, ethoxyl, propoxyl, and phenoxyl; a cyano group; a nitro group; and halogens, such as fluorine and chlorine. The substituent of the alkyl group can be methyl, ethyl, n-propyl, isopropyl, n-butyl, isobutyl, sec-butyl, and tert-butyl. In particular, methyl or tert-butyl may be selected.

The organic compound according to the present embodiment includes a basic skeleton having both a condensed five-membered structure and a spiro structure. The basic skeleton mentioned herein refers to a skeleton formed only by annelation. More specifically, the basic skeleton mentioned herein refers to the portion expressed by rings excluded R₁ to R₄ in General Formula (1).

One of the features of the organic compound of the embodiments of the present invention is that it has a spiro structure having the following features. The spiro structure includes intersecting planes a and b shown in the above General Formula (1). More specifically, planes a and b are orthogonal to each other. This structure does not have a rotatable bond.

The dihedral angle between planes a and b obtained by molecular orbital calculation was 89.9°. Hence, the two planes are perpendicular to each other. The structure in which the two planes form a right angle is the most stable. The molecular orbital calculation was performed as below. All the molecular orbital calculations in the present embodiment were performed according to the same method. For the molecular orbital calculation, DFT basis function 6-31+G(d) was applied according to Gaussian 03 (Gaussian 03, Revision D. 01, M. J. Frisch, G. W. Trucks, H. B. Schlegel, G. E. Scuseria, M. A. Robb, J. R. Cheeseman, J. A. Montgomery, Jr., T. Vreven, K. N. Kudin, J. C. Burant, J. M. Millam, S. S. Iyengar, J. Tomasi, V. Barone, B. Mennucci, M. Cossi, G. Scalmani, N. Rega, G. A. Petersson, H. Nakatsuji, M. Hada, M. Ehara, K. Toyota, R. Fukuda, J. Hasegawa, M. Ishida, T. Nakajima, Y. Honda, O. Kitao, H. Nakai, M. Klene, X. Li, J. E. Knox, H. P. Hratchian, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, P. Y. Ayala, K. Morokuma, G. A. Voth, P. Salvador, J. J. Dannenberg, V. G. Zakrzewski, S. Dapprich, A. D. Daniels, M. C. Strain, O. Farkas, D. K. Malick, A. D. Rabuck, K. Raghavachari, J. B. Foresman, J. V. Ortiz, Q. Cui, A. G. Baboul, S. Clifford, J. Cioslowski, B. B. Stefanov, G. Liu, A. Liashenko, P. Piskorz, I. Komaromi, R. L. Martin, D. J. Fox, T. Keith, M. A. Al-Laham, C. Y. Peng, A. Nanayakkara, M. Challacombe, P. M. W. Gill, B. Johnson, W. Chen, M. W. Wong, C. Gonzalez, and J. A. Pople, Gaussian, Inc., Wallingford Conn., 2004). Although it is known that this calculation gives an error, it is useful in designing molecules.

The organic compound according to the present embodiment includes plane a shown in General Formula (1). Plane a has a larger conjugation length than fluoranthene, and has a higher oscillator strength and a higher quantum yield accordingly. It is known that a high oscillator strength leads to a high quantum yield.

Since the basic skeleton of the organic compound of the present embodiment does not have a rotatable bond, thermal motion consuming energy does not occur. This may be a reason why the quantum yield is high.

For the above-described reasons, the organic compound of the present embodiment can be used as a luminescent material for organic light-emitting elements. The use of the organic compound of the present embodiment imparts a high quantum yield to the luminescent material and reduces molecular aggregation to prevent concentration quenching.

The organic compound of the present embodiment can be used particularly as the guest material of a luminescent layer containing a host material and a guest material. The host material refers to the material having the highest weight ratio in a luminescent layer. The guest material refers to a material having a lower weight ratio than the host material and is the most luminous material in the luminescent layer.

The organic compound of the present embodiment can be used as a guest material of a blue light-emitting element. By adjusting the emission wavelength of the guest material, the organic compound can be used for green and red light-emitting elements.

The organic compound of the present embodiment may be used in any layer of the organic light-emitting element, in addition to the luminescent layer. For example, the organic compound may be used in a hole injection layer, a hole transport layer, an electron transport layer, an electron injection layer, or a hole exciton blocking layer.

Examples of the organic compound include, but are not limited to, the following:

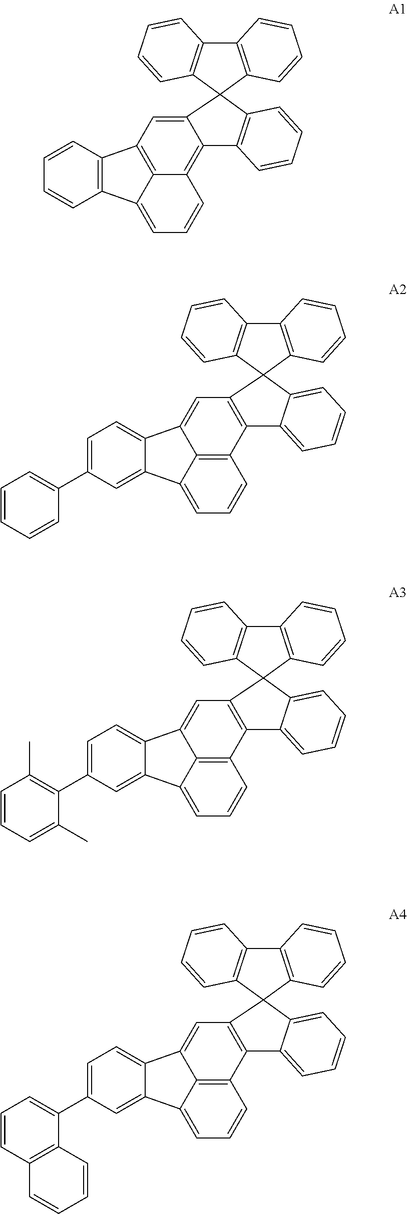

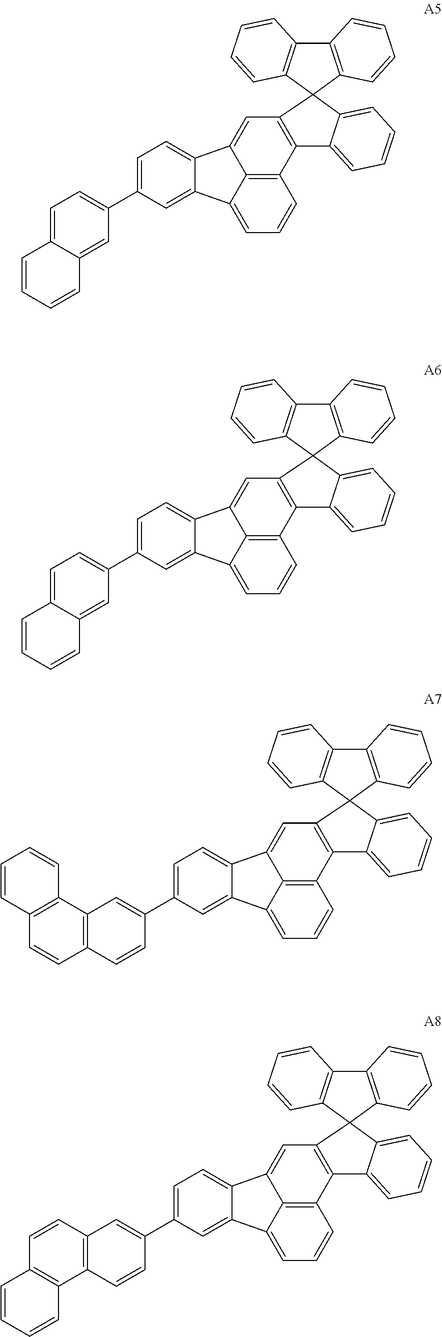

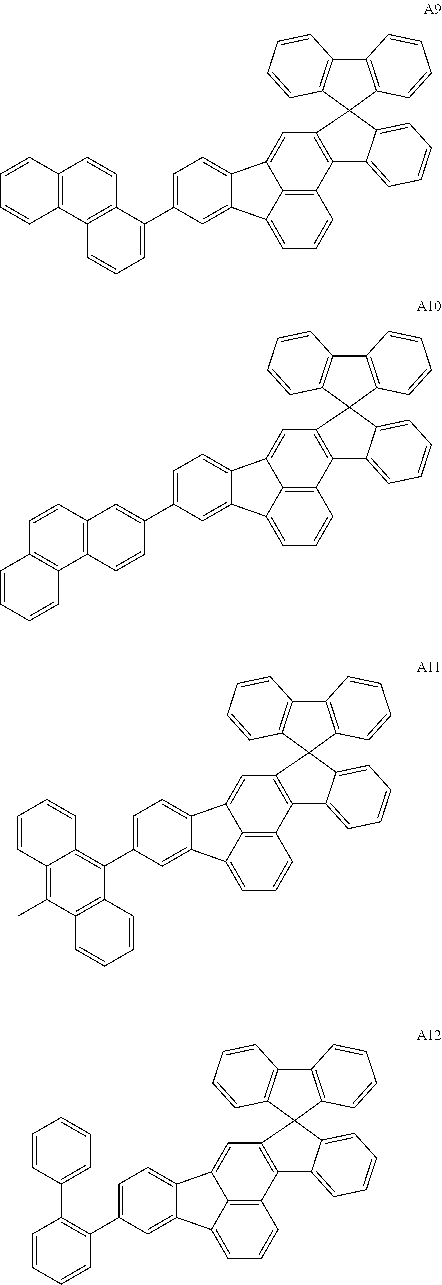

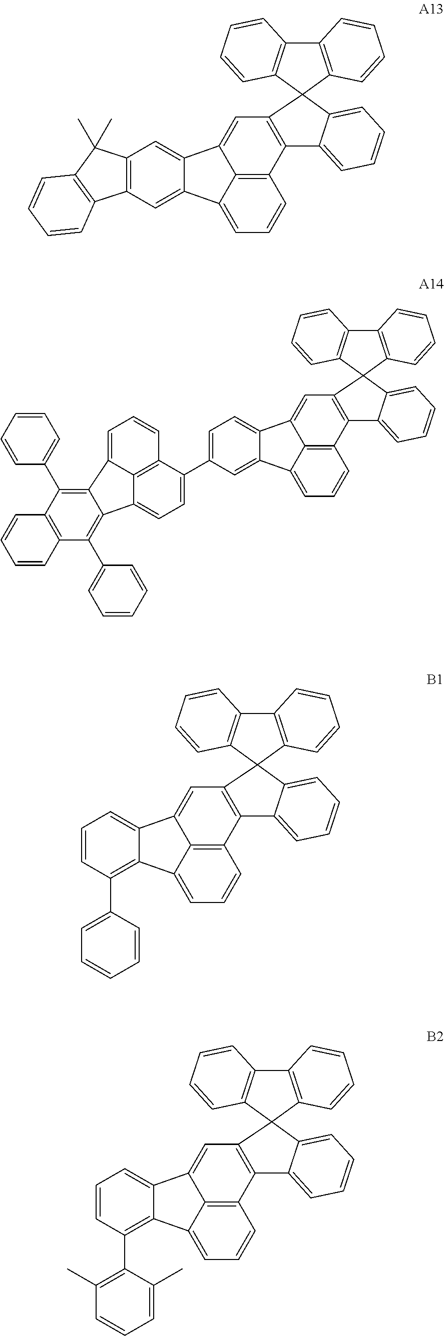

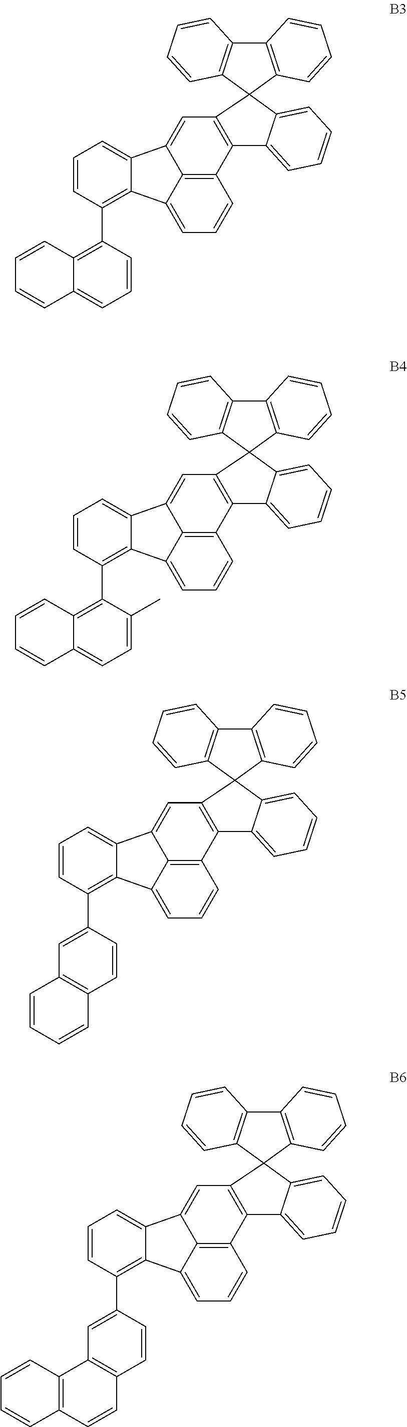

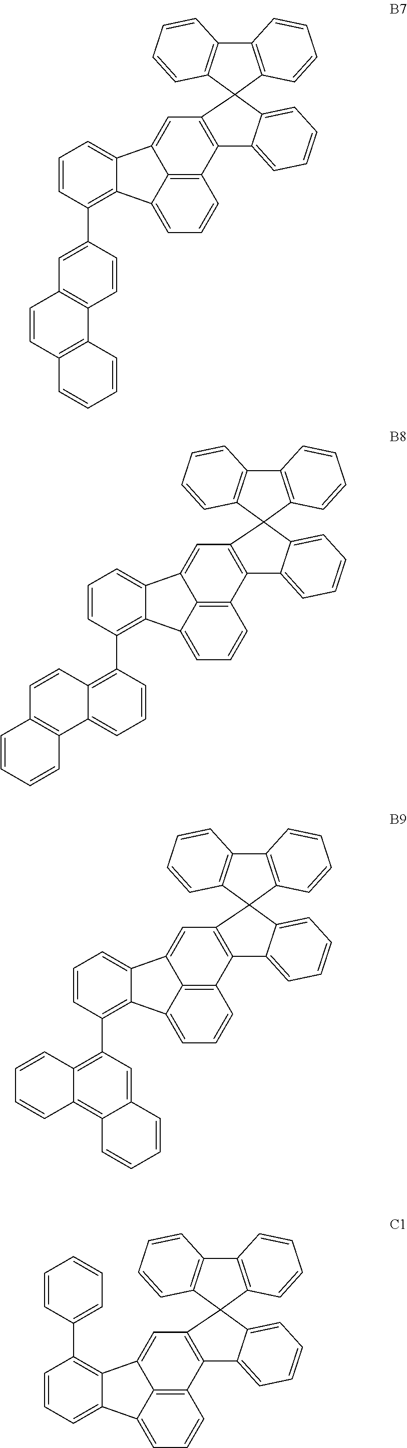

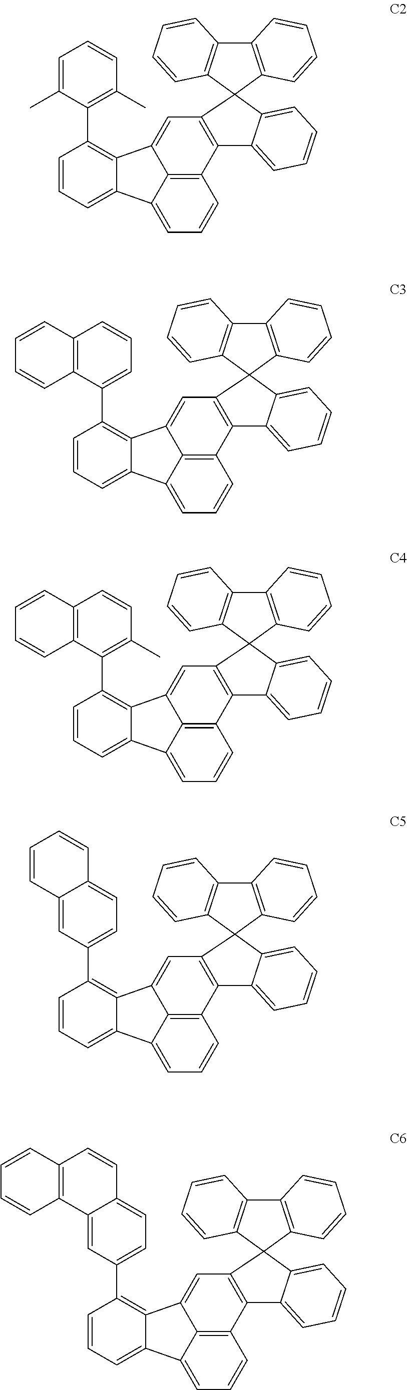

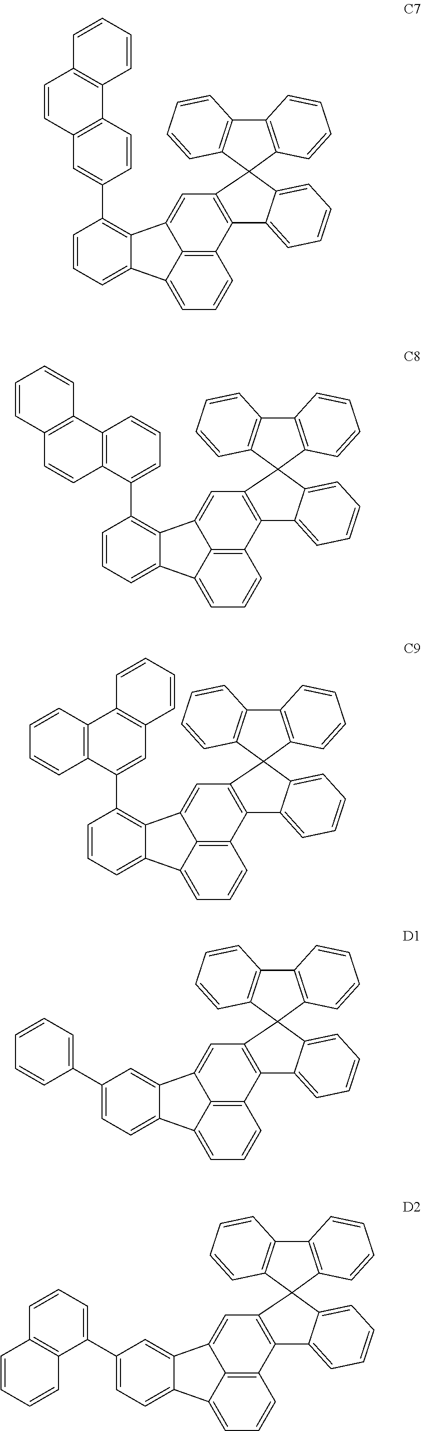

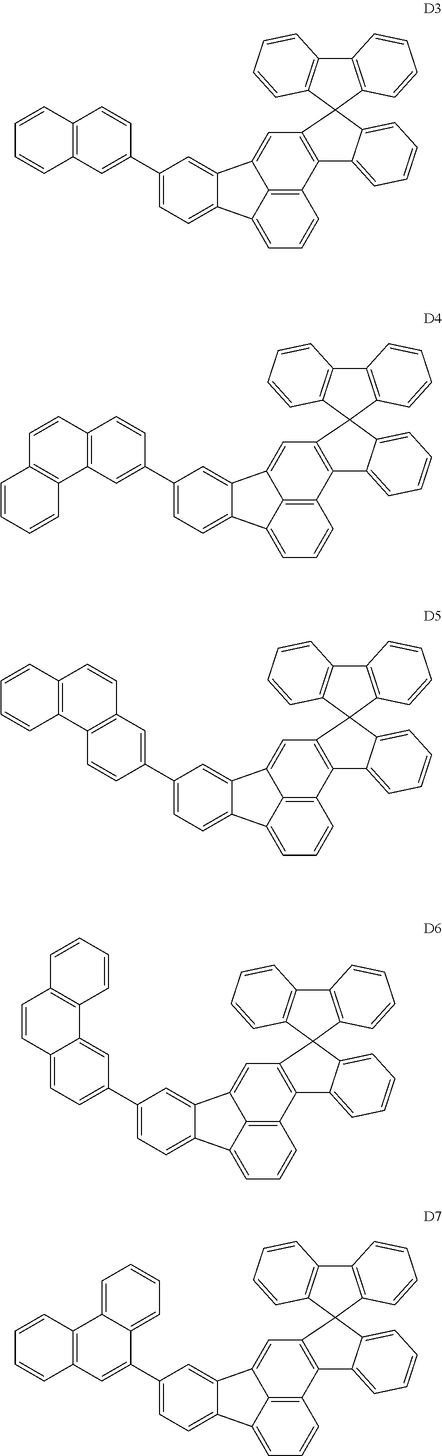

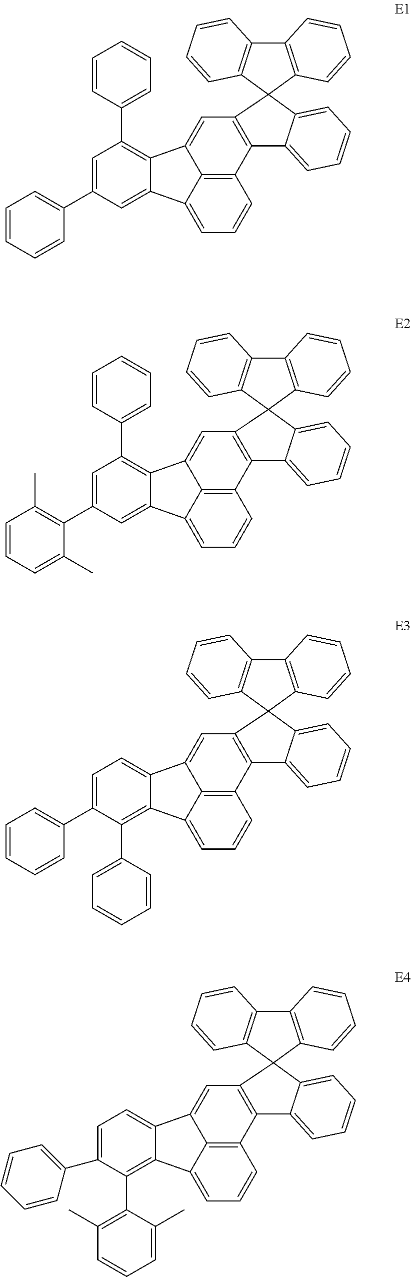

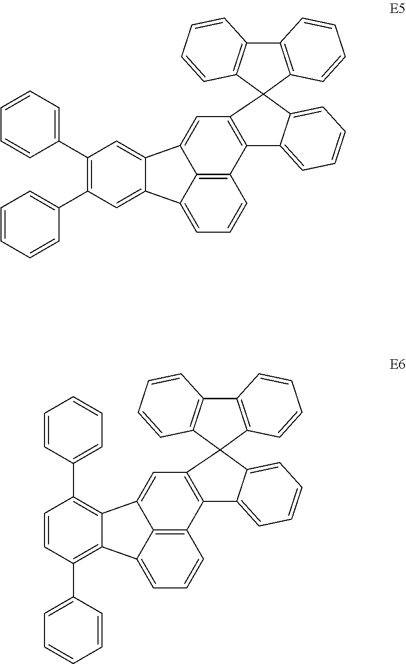

The above listed compounds include a basic skeleton having an aryl group. The basic skeleton may have an alkyl group (not shown), if necessary.

The compounds having a substituent at the position of R₁ of General Formula (1), that is, Compounds B1 to B9, can further inhibit molecular aggregation.

Organic compounds expressed by General Formula (2) can be the most advantageous of the above-listed compounds.

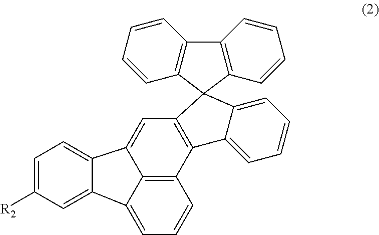

In General Formula (2), R₂ represents hydrogen, an alkyl group or an aryl group. The alkyl group is selected from the group consisting of methyl, ethyl, n-propyl, isopropyl, n-butyl, isobutyl, sec-butyl, and tert-butyl. These alkyl groups act as sterically hindering groups to inhibit molecular aggregation. Among these groups, tert-butyl is effective. The aryl group is selected from the group consisting of phenyl, naphthyl, biphenyl, phenanthrenyl, fluorenyl, and benzofluoranthenyl. These aryl groups can change the emission wavelength and enhance the quantum yield.

In particular, R₂ in General Formula (2) can be an aryl group. Organic compounds having an aryl group at this position have a high oscillator strength, and accordingly have a high quantum yield. Compounds A1 to A14 correspond to this type.

Oscillator strengths of organic compounds were obtained by molecular orbital calculation. The results show that the compounds having a substituent at the R₂ position have higher oscillator strength than compounds having a substituent at other positions. A high oscillator strength leads to a high quantum yield. The calculated oscillator strengths of some of the above listed compounds are shown in Table 1.

TABLE 1

| Compound | Oscillator strength |
|---|---|
| A1 | 0.18 |
| A2 | 0.34 |
| A6 | 0.42 |
| A14 | 0.58 |
| B1 | 0.24 |
| C1 | 0.1 |
| D1 | 0.23 |
| Fluoranthene | 0.0038 |

Table 1 shows that the organic compounds according to the present embodiment have higher oscillator strengths than fluoranthene. The oscillator strength can be further increased by introducing an aryl group to the R₂ position, as shown by Compounds A2, A6 and A14. In addition, by introducing an aryl group having a higher quantum yield, such as fluorenyl or benzofluoranthenyl, to the R₂ to R₄ positions of a compound expressed by General Formula (1), the resulting compound can exhibit still higher quantum yield.

### Synthesizing Process

The organic compound of the present embodiment can be synthesized in, but is not limited to, the following process. In the process, Compound F1 used as a raw material can be synthesized referring to Organic Electronics 9 (2008) 522-532. Intermediate F2 can be synthesized, for example, by allowing Compound F1 to react with 4,4,5,5-tetramethyl-[1,3,2]dioxaborane in a solvent of toluene in the presence of triethylamine and a catalyst of [1,3-bis(diphenylphosphino)propane]dichloronickel (Ni(dppp)Cl₂).

Intermediate F3 can be synthesized, for example, by allowing Intermediate F2 to react with a corresponding halogen-substituted compound in a mixed solvent of toluene and distilled water in the presence of sodium carbonate and a catalyst of tetrakis(triphenylphosphine)Palladium (Pd (PPh₃)₄).

Intermediate F4 can be synthesized, for example, by allowing Intermediate F3 to react in a solvent of N,N-dimethylformamide (DMF), in the presence of diazabicycloundecene (DBU), LiCl and a catalyst of bis(triphenylphosphine)palladiumchloride (Pd(PPh₃)₂Cl₂).

Compound F4 is cross-coupled with an aryl boronic acid or an aryl halide with pinacolborane to synthesize Compound F5 formed by introducing a substituted or unsubstituted aryl to the R₂ position of a compound expressed by General Formula (2). Examples of the synthesis of Compound F5 are shown in Table 2.

For synthesizing Intermediate F3, other halogen-substituted compounds having halogens at different positions can be produced by using different halogen-substituted benzenes. The resulting compounds are subjected to cross coupling to synthesize organic compounds expressed by General Formula (1) having various aryl groups at the R₁, R₃ and/or R₄ position. By using corresponding halogen-substituted benzenes, all the compounds listed above can be synthesized.

### Examples of Synthesis

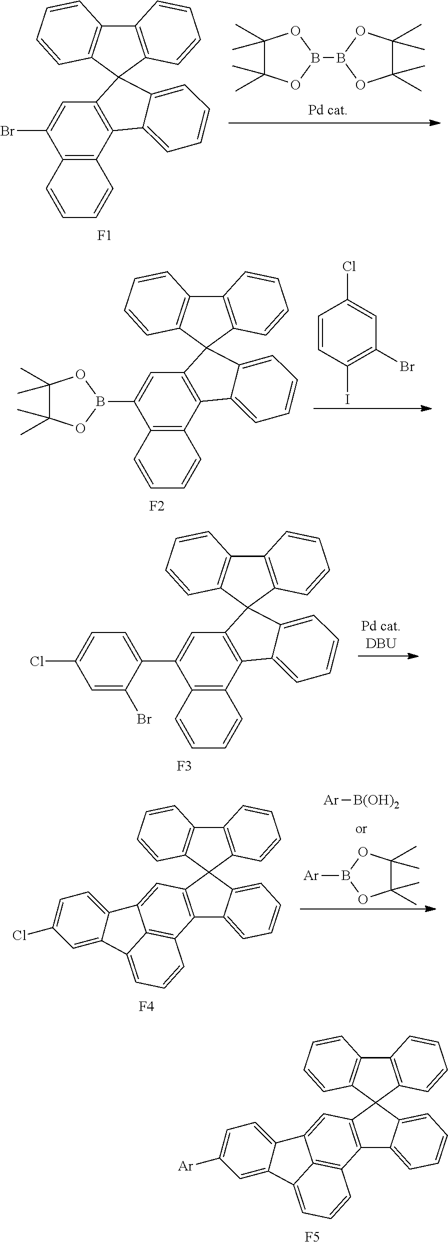

TABLE 2

| | 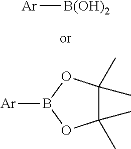 | Synthesized compound |
|---|---|---|
| Synthesis example 1 | 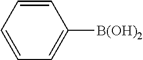 | 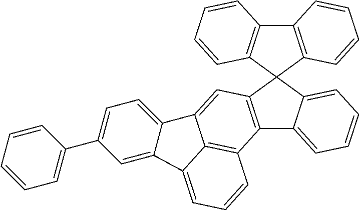 |
| Synthesis example 2 | 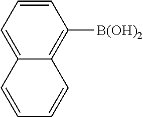 | 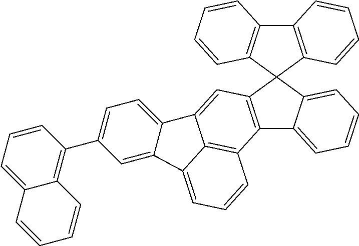 |
| Synthesis example 3 | 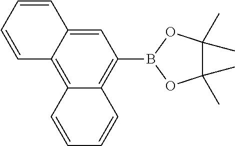 | 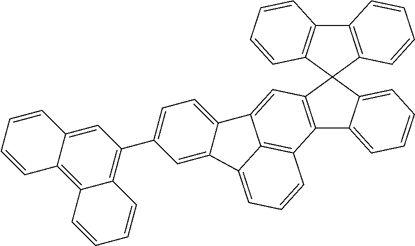 |
| Synthesis example 4 | 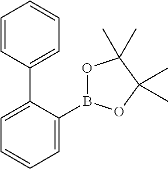 | 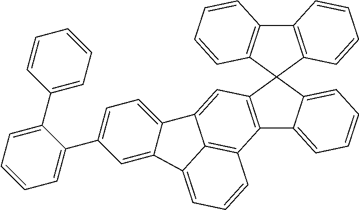 |
| Synthesis example 5 | 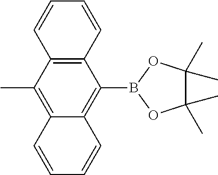 | 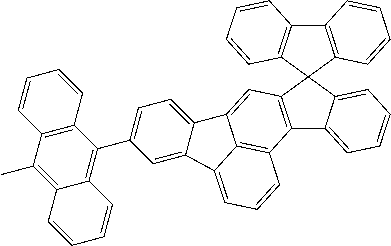 |

### Organic Light-Emitting Element

An organic light-emitting element according to an embodiment will now be described. The organic light-emitting element includes a pair of electrodes (anode and cathode) and an organic compound layer between the electrodes. The organic compound layer contains an organic compound expressed by General Formula (1). The organic compound layer may have a multilayer structure. The layers of the multilayer structure include a hole injection layer, a hole transport layer, a luminescent layer, a hole blocking layer, an exciton blocking layer, an electron transport layer, and an electron injection layer. The organic compound layer may be defined by an appropriate combination of these layers. If the organic compound according to an embodiment of the present invention is used as a guest material, the proportion of the guest material can be 0.1% to 30% by mass, such as 0.5% to 10% by mass, relative to the host material.

The present inventors have found that an organic light-emitting element including a luminescent layer containing the organic compound of an embodiment of the present invention as a host material or a guest material, particularly as a guest material, efficiently emits bright light and has high durability.

The organic light-emitting element of the present embodiment may contain a known low-molecular-weight or polymeric hole injecting or transporting material, or an electron injecting or transporting material being a host or guest material, together with the organic compound of an embodiment of the invention.

Examples of these materials are shown below. The hole injecting material or hole transporting material can have a high hole mobility. Examples of the low-molecular-weight or polymeric hole injecting or transporting materials include, but are not limited to, triarylamine derivatives, phenylenediamine derivatives, stilbene derivatives, phthalocyanine derivatives, porphyrin derivatives, poly(vinyl carbazole), poly(thiophene), and other conductive polymers.

Table 3 shows structural formulas of exemplary host materials. The host material may be a derivative of the compounds expressed by the structural formulas shown in Table 3. Other host materials include, but are not limited to, annelated compounds, such as fluorene derivatives, naphthalene derivatives, anthracene derivatives, pyrene derivatives, carbazole derivatives quinoxaline derivatives, and quinoline derivatives; organic aluminum complexes, such as tris(8-quinolinolate) aluminum; organic zinc complexes; and other polymer derivatives, such as triphenylamine derivatives, poly(fluorene) derivatives, and poly(phenylene) derivatives.

TABLE 3

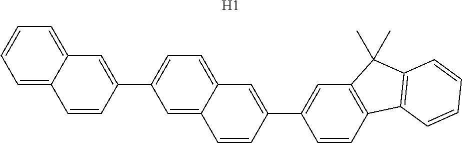

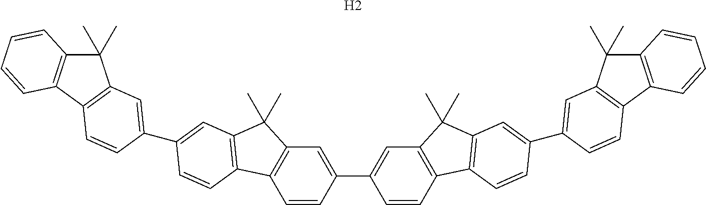

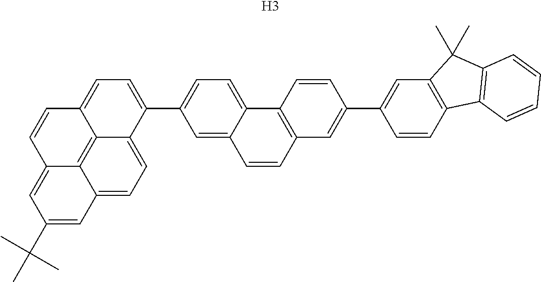

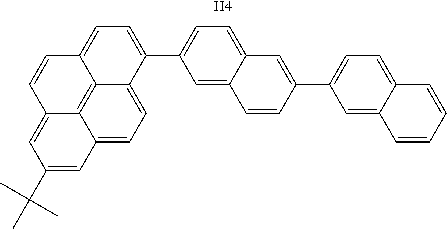

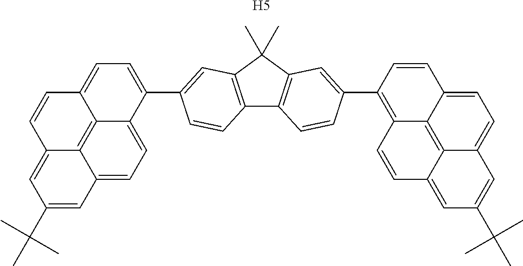

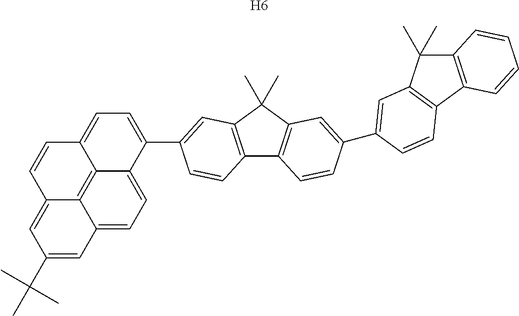

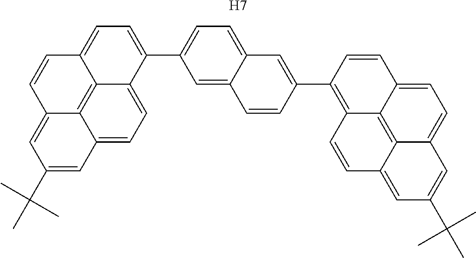

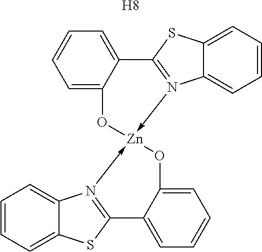

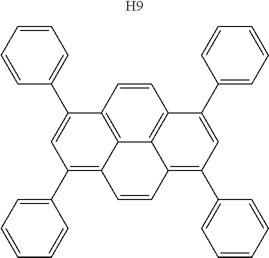

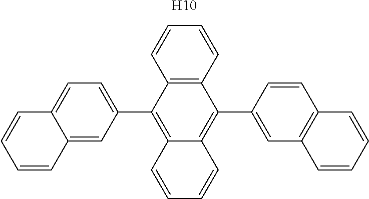

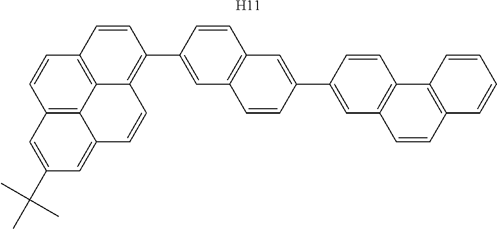

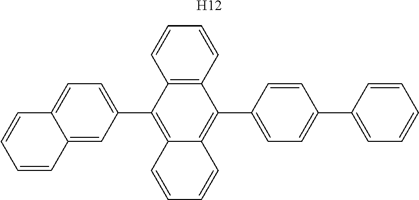

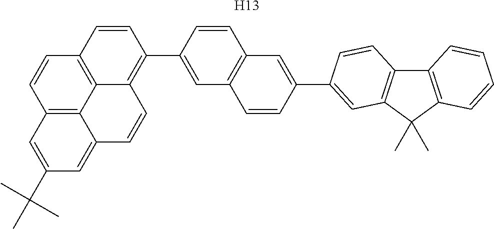

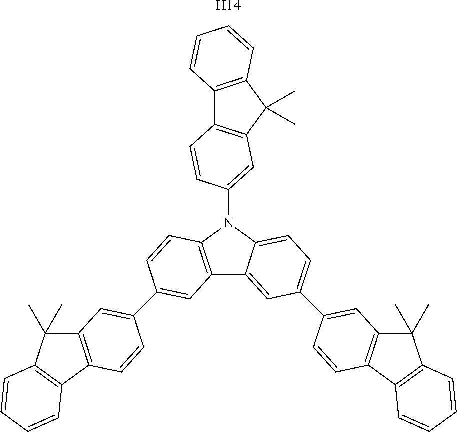

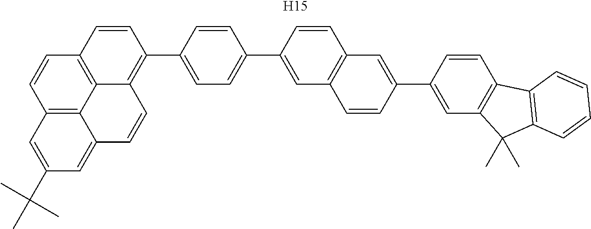

The electron injecting or transporting material is selected in view of the balance with the hole mobility and other properties of the hole injecting or transporting material. Examples of the electron injecting or transporting material include, but are not limited to, oxadiazole derivatives, oxazole derivatives, pyrazine derivatives, triazole derivatives, triazine derivatives, quinoline derivatives, quinoxaline derivatives, phenanthroline derivatives, and organic aluminum complexes.

A compound having a work function as high as possible can be used as the material of the anode. Examples of such a material include elemental metals and their alloys, such as gold, platinum, silver, copper, nickel, palladium, cobalt, selenium, vanadium and tungsten; and metal oxides, such as tin oxide, zinc oxide, indium oxide, indium tin oxide (ITO), and zinc indium oxide. Conductive polymers may be used, such as polyaniline, polypyrrole, and polythiophene. These anode materials may be used singly or in combination. The anode may have a single-layer structure or a multilayer structure.

On the other hand, a material having a low work function can be suitably used for the cathode. Examples of the cathode material include alkali metals, such as lithium; alkaline-earth metals, such as calcium; and other elemental metals, such as aluminum, titanium, manganese, silver, lead, and chromium. In addition, alloys of these elemental metals may be used. Such alloys include magnesium-silver, aluminum-lithium, and aluminum-magnesium. A metal oxide, such as indium tin oxide (ITO), may be used. These cathode materials may be used singly or in combination. The cathode may have a single-layer structure or a multilayer structure.

In the organic light-emitting element of the present embodiment, the layer containing the organic compound of an embodiment of the invention and other layers are formed in the following process. In general, each layer can be formed by vacuum vapor deposition, ionized vapor deposition, sputtering, or plasma or solution coating, such as spin coating or dipping, a cast method, a Langmuir-Blodgett (LB) method, or an ink jet method. Layers formed by vacuum vapor deposition, solution coating or the like are difficult to crystallize and superior in temporal stability. For coating, an appropriate binder resin may be used in combination.

Examples of the binder resin include, but are not limited to, polyvinylcarbazole resin, polycarbonate resin, polyester resin, ABS resin, acrylic resin, polyimide, phenol resin, epoxy resin, silicone resin, and urea resin. These binder resins may be used in a single form of homopolymer or copolymer, or in a form of mixture. A known additive, such as plasticizer, antioxidant or ultraviolet light adsorbent, may be used in combination with the binder resin.

### Application of Organic Light-Emitting Element

The organic light-emitting element of the present embodiment can be used for a display device or an illuminating device. In addition, the organic light-emitting element can be used for an exposure light source of an electrophotographic image forming apparatus or a backlight of a liquid crystal display device. The display device according to an embodiment of the present invention includes a display portion including the organic light-emitting element of an embodiment of the invention. The display portion includes a plurality of pixels. The pixel includes the organic light-emitting element of the present embodiment, and a TFT element as a switching element controlling the brightness of emitted light. The anode or the cathode of the organic light-emitting element is connected to the drain electrode or the source electrode of the TFT element. The display device can be used as an image display device of a PC or the like.

The display device may be used in an image output apparatus that includes an image input portion to which image information is inputted from an area CCD, a linear CCD, a memory card or the like, and a display portion to which an image is inputted according to the inputted image information. The display device may be used as a display portion of an image pickup device or an ink jet printer. In this instance, the display device has both an image output function on which an image is displayed according to image information inputted from the outside, and an input function as an operation panel from which information is inputted to process the image. Also, the display device may be used as a display portion of a multifunction printer.

The display device using the organic light-emitting element according to an embodiment of the invention will be further described with reference to the FIGURE. The FIGURE is a schematic sectional view of a display device that includes organic light-emitting elements according to an embodiment of the present invention, and TFT elements as switching elements connected to the respective organic light-emitting elements. The FIGURE shows two sets of organic light-emitting elements and TFT elements. This structure will now be described in detail.

The display device shown in the FIGURE includes a substrate 1 made of glass or the like, and a moisture-proof layer 2 covering the substrate to protect TFT elements 8 or organic compound layers 12. Reference numeral 3 designates a metal gate electrode 3. Reference numeral 4 designates a gate insulation film 4, and reference numeral 5 designates a semiconductor layer.

Each TFT element 8 includes a semiconductor layer 5, a drain electrode 6, and a source electrode 7. An insulating layer 9 is disposed over the TFT elements 8. The source electrode 7 is connected to the anode 11 of the corresponding organic light-emitting element through a contact hole 10. The display device is not limited to this structure. Either the anode or the cathode 13 can be connected to either source electrode 7 or the drain electrode 6 of the TFT element 8.

Although the organic compound layer 12 is shown as a single layer in the FIGURE, it may include a plurality of layers. Furthermore, a first protective layer 14 and a second protective layer 15 are disposed over the cathode 13 to prevent the degradation of the organic light-emitting element.

The switching element is not particularly limited in material, and may be a single-crystal silicon element, a metal-insulator-metal (MIM) element, or an amorphous silicon (a-Si) element.

### Example

Examples of the present invention will now be described, but the invention is not limited to the following Example.

### Production of Compound A1

Compound A1, which is one of the organic compounds of the embodiments of the invention, was synthesized in the following process.

### Preparation of Intermediate F7

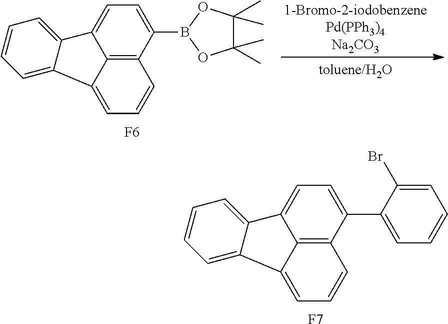

In a nitrogen atmosphere, the following three compounds were dissolved in 12 mL of toluene, and solution of 0.85 g (8.07 mmol) of sodium carbonate in 4 mL of distilled water was added. The reaction liquid was heated with stirring for 24 hours in a silicone oil bath heated to 80° C.

Three compounds dissolved in toluene:

1-Bromo-2-iodebenzene, 0.948 g (3.36 mmol)

Compound F6, 1.00 g (3.05 mmol)

Tetrakis(triphenylphosphine)palladium, 0.212 g (0.183 mmol)

After cooling to room temperature, water and toluene were added to the reaction liquid to separate out the organic phase. The water phase was further subjected to extraction with toluene (twice), and the extract was added to the separated organic phase. The organic phase was washed with saturated saline and then dried with sodium sulfate. The solvent was evaporated, and the residue was purified by silica gel column chromatography (mobile phase, chloroform:heptane=1:10) to yield 0.179 g of Intermediate F7 (yield: 16%).

### Synthesis of Compound A1

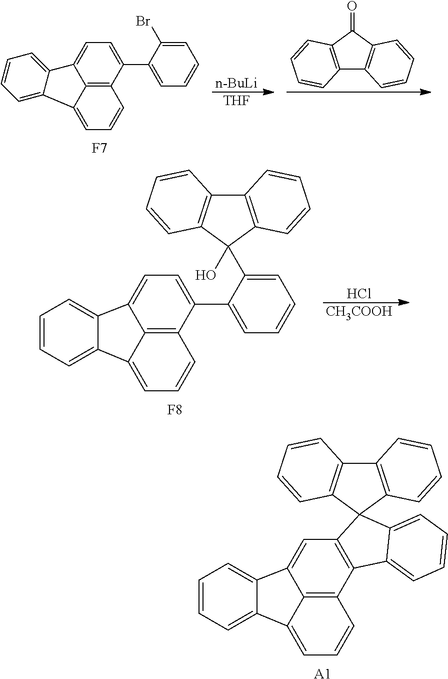

In a nitrogen atmosphere, 0.179 g (0.501 mmol) of Intermediate F7 was dissolved in 2 mL of tetrahydrofuran, and the solution was cooled to −78° C. Then, 0.376 mL (0.601 mmol) of n-butyl lithium was slowly dropped to the solution, followed by stirring for 1 hour. To the mixture was slowly added 0.081 g (0.451 mmol) of 9-fluorenone dissolved in 1.5 mL of tetrahydrofuran at −78° C., and the mixture was stirred for 4 hours while being gradually returned to room temperature.

Water was added to the reaction liquid, and the reaction product was extracted with chloroform and dried with sodium sulfate. After evaporation of the solvent, 2.5 mL of acetic acid and 0.2 mL of dilute hydrochloric acid were added to the residue, followed by refluxing for 6 hours. An aqueous solution of sodium hydrogencarbonate was added to the reaction product. The product was extracted with chloroform and dried with sodium sulfate. The residue was purified by silica gel column chromatography (mobile phase, chloroform:heptane=1:5) to yield 0.035 g of Compound A1 (yield: 16%).

The resulting compound was subjected to MALDI-TOF MS (matrix assisted laser desorption ionization-Time of Flight Mass Spectrometry), and the molecular ion peak (M+) of Compound A1 was confirmed at 440.0.

In addition, the structure of Compound A1 was confirmed by ¹H-NMR analysis.

¹H-NMR (CDCl₃, 600 MHz) δ (ppm): 8.66 (1H, d, J=8.40 Hz), 8.40 (1H, d, J=7.80 Hz), 7.97 (1H, d, J=6.60 Hz), 7.92 (2H, d, J=7.80 Hz), 7.84 (1H, d, J=7.20 Hz), 7.80 (1H, t, J=7.20, 7.80 Hz), 7.57 (1H, d, J=7.20 Hz), 7.46 (1H, t, J=7.20, 7.80 Hz), 7.41 (2H, t, J=7.20, 7.80 Hz), 7.29-7.21 (2H, m), 7.19 (1H, s), 7.14 (1H, t, J=7.20, 7.80 Hz), 7.11 (2H, t, J=7.20, 7.80 Hz), 6.81 (1H, d, J=7.20 Hz), 6.77 (2H, d, J=7.20 Hz)

The photoluminescence (PL) spectrum of a dilute solution of Compound A1 in toluene was measured at an excitation wavelength of 350 nm using F-4500 manufactured by Hitachi. An emission spectrum of greenish-blue light having the highest intensity at 479 nm was observed. The emission quantum yield of the dilute solution was measured using an absolute quantum yield measurement system (C9920-02, manufactured by Hamamatsu Photonics). The result of the measurement was 0.66.

### Organic Light-Emitting Element Using Compound A1

It is believed that Compound A1 can be used for an organic light-emitting element. For example, the organic light-emitting element may have a structure including an anode, a hole transport layer, a luminescent layer, a hole exciton blocking layer, an electron transport layer and a cathode in that order. The layers can be as follows:

Hole transport layer (30 nm), Compound G1

Luminescent layer (30 nm), host: Compound G2, guest: Compound A1

Electron transport layer (30 nm), Compound G3

Metal electrode layer 1 (1 nm), LiF

Metal electrode layer 2 (100 nm), Al

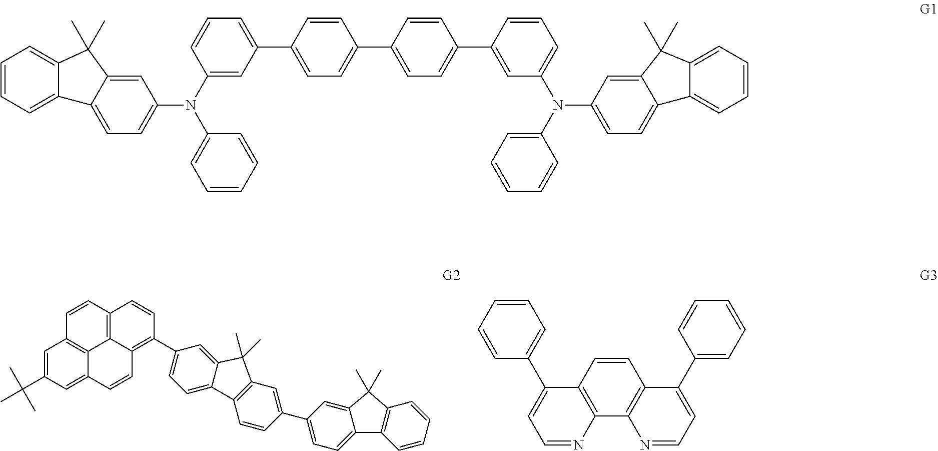

It is believed that the organic light-emitting element having such a structure can emit light.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-030453 filed Month Feb. 15, 2010 hereby incorporated by reference herein in its entirety.

What is claimed is:

1. An organic compound expressed by General Formula (1): 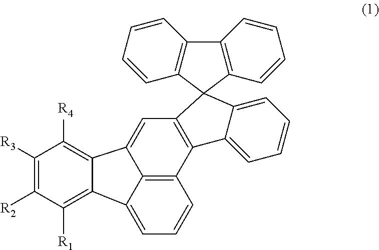
wherein R₁ to R₄ are each selected from the group consisting of hydrogen, alkyl groups and aryl groups, wherein the alkyl groups consist of methyl, ethyl, n-propyl, isopropyl, n-butyl, isobutyl, sec-butyl, and tert-butyl, and the aryl groups consist of phenyl, biphenyl, terphenyl, naphthyl, phenanthrenyl, anthracenyl, fluoranthenyl, benzofluoranthenyl, and fluorenyl, and wherein the alkyl groups and the aryl groups may have a substituent selected from the group consisting of alkyl, aralkyl, aryl, heterocycle, amino, alkoxyl, cyano, and halogens.

2. An organic light-emitting element comprising:
a pair of electrodes; and
an organic compound layer disposed between the electrodes, the organic compound layer containing the organic compound as set forth in claim 1.

3. The organic light-emitting element according to claim 2, wherein the organic compound layer acts as a luminescent layer.

4. A display device comprising:
a plurality of pixels, each including an organic light-emitting element as set forth in claim 2 and a switching element connected to the organic light-emitting element.

5. An image output apparatus comprising:
an image input portion to which an image is inputted; and
a display portion on which an image is outputted, the display portion including a plurality of pixels, each including the organic light-emitting element as set forth in claim 2 and a switching element connected to the organic light-emitting element.
